(19) United States
(12) Patent Application Publication
(10) Pub. No.: US 2008/0183250 A1
(43) Pub. Date: Jul. 31, 2008

(54) Compositions and methods for treating or preventing skin inflammation via restoration of skin barrier function

(76) Inventors: Hanafi Tanojo, Palo Alto, CA (US); Xinfan Huang, Menlo Park, CA (US)

Correspondence Address: WILSON SONSINI GOODRICH & ROSATI, 650 PAGE MILL ROAD, PALO ALTO, CA, 94304-1050, US

(21) Appl. No.: 11/985,806

(22) Filed: Nov. 16, 2007

Related U.S. Application Data
(63) Continuation-in-part of application No. 11/275,994, filed on Feb. 8, 2006, now Pat. No. 7,300,649.
(60) Provisional application No. 60/652,196, filed on Feb. 11, 2005.

(51) Int. Cl. A61K 33/20; A61P 17/00; A61N 5/06

(52) U.S. Cl. 607/88; 424/663

(57) ABSTRACT

Compositions, kits and methods are provided for restoring skin barrier function to skin of a subject after a medical or a cosmetic procedure. In general, a composition containing divalent cations such as calcium ions and/or magnesium ions in a physiologically acceptable medium can be used to treat, inhibit or prevent inflammation of skin post medical, demertological, or cosmetic procedure such as chemical peeling, laser skin resurfacing, microdermabrasion, plasma energy treatment, radiofrequency treatment, hair or vein removal or lightening, skin treatment using light energy or photofacial, thereby treating, inhibiting or preventing post-inflammatory hyperpigmentation of the skin or other skin disorders.

### CROSS REFERENCE

This application is a continuation-in-part of U.S. application Ser. No. 11/275,994, filed Feb. 8, 2006, which claims the benefit of U.S. Provisional Application No. 60/652,196, filed Feb. 11, 2005, which applications are incorporated herein by reference in their entirety.

### BACKGROUND OF THE INVENTION

The skin serves numerous functions but its primary function is as a protective layer or barrier. The most important role of the skin for terrestrial animals is to protect the water-rich internal organs from the dry environment. This cutaneous barrier function of the skin resides in the upper most thin layer (approximately 10-20 μm in humans) called stratum corneum. The water impermeability of this layer is 1000 times-high than that of other membranes of living organisms. Potts & Francoeur (1991) J. Invest. Dermatol. 96; 495-499.

The stratum corneum is composed of two components, i.e., protein-rich nonviable cells and intercellular lipid domains. Elias et al. (1993) Curr. Opin. Dermatol. 231-237. The lipid molecule in the intercellular domain form a bilayer structure. The water impermeability is due to the conformation of the lipid molecules and also the order of the deal cells. Denda et al. (1994) Arch. Dermatol. Res. 286:41-46. Because of this specific “brick and mortar” structure, the stratum corneum shows high water impermeability.

The uppermost layer of the skin, called epidermis, is mainly constructed of keratinocytes. The epidermis is in a constant state of self-replacement. At the bottom layer, keratinocyte stem cells divide into daughter cells, which are displaced outward, and which differentiate through successive overlying layers to enter the stratum corneum. Then, the keratinocytes die (apoptosis) and their cellular organella and cytoplasm disappear during the final process of differentiation. Intercellular lipids are primarily generated from exocytosis of lipid-containing granules called lamellar bodies, during the terminal differentiation. The secreted lipids spread over the intercellular domains and form a bilayer structure. Elias et al. (1993), supra.

Ionic signals play important role in the homestatic mechanism of the epidermal barrier function. Lee et al. (1992) J. Clin. Invest. 89:530-538. In normal skin, calcium is localized with high concentration in the epidermal granular layer, i.e., the uppermost layer of the epidermis, just below the stratum corneum. On the contrary, the concentration potassium is the highest in the spinous layer, i.e., middle of the epidermis, and the lowest in the granular layer.

Calcium is a universal messenger, even in simple organisms and plants. The combination of its ionic radius and double charge may allow it tighter binding to receptors to the exclusion of other ions such as magnesium, leading to strong, specific binding. Carafoli & Penniston (1985) The Calcium Signal. Sci. Am. 253:70-78. The specificity enables cells to form special receptors to assess signals from calcium. For many parts of the body, Ca²⁺ often acts as a second messenger in a manner similar to cAMP. In skin, calcium can provide signals for the cells, either extracellular or intracellular (in the cytosol). The extra- and intracellular signaling may be connected to each other, but may also act separately. It has been found that intracellular Ca²⁺ increases with raised extracellular Ca²⁺. This implies that increased intracellular Ca²⁺ is the actual signal to trigger keratinocytes differentiation. Tanojo & Maibach (1999) in Percutaneous Absorption, 3^{rd} Ed., Bronaugh & Maibach, ed., Marcel Dekker, NY, pp. 939-950.

As Ca²⁺ cannot be metabolized like other second-messenger molecules, cells tightly regulate intracelular levels thorough numerous binding and specialized extrusion proteins. Clapham (1995) Cell 80:259-268. The concentration of calcium in extracellular spaces (generally ˜1.5 mM) is four orders of magnitude higher than in the cytosol (˜0.1 μM). In excitable cells, for example, muscle cells, the extracellular concentration of calcium must be closely regulated to keep it at its normal level of 1.5 mM, so that it cannot accidentally trigger the muscle contraction, the transmission of nerve impulses, and blood clotting. In other cells, including keratinocytes, the extracellular level is maintained in a specific equilibrium with the intracellular concentrations.

As described above, there is a high calcium gradient between extra- and intracellular domains of keratinocytes, which requires tight regulation. Moreover, a calcium gradient is present within the epidermis, with higher quantities of Ca²⁺ in the upper than the lower epidermis. Menon et al. (1985) J. Invest. Dermatol. 102:789-795. Ca²⁺ concentration increases steadily from the basal region to stratum corneum, which this is not the case with other ions. Forslind et al (1995) Scanning Microsc. 9:1011-1026. Such a gradient is not observed in skin abnormalities related to the formation of abnormal barrier function, such as psoriasis. Menon & Elias (1991) Arch. Dermatol. 127:57-63. It has been reported that disruption of the skin barrier with acetone treatment or tape stripping depletes Ca²⁺ from the upper epidermis, resulting in the loss of the Ca²⁺ gradient. This is due to accelerated water transit that leads to the increased passive loss of Ca²⁺ into and through the stratum corneum. Mao-Qiang et al. (1997) Exp. Dermatol. 6:36-40.

In summary, calcium ions play an important role in the homeostasis of skin barrier. A change in the barrier will change the calcium ion gradient in skin and lead to barrier repair process. A severe change might lead to a high degree of calcium signaling, which may induce the activation of various processes, from increased synthesis of skin components or messengers to the inflammatory reactions. Thus, there exists a need for compositions and methods for activating the barrier repair process to restore normal barrier function to skin adversely affected by environmental elements or pathological conditions.

### SUMMARY OF THE INVENTION

Compositions, systems, kits and methods are provided for restoring skin barrier function to skin exposed to environmental elements, undergone a medical or cosmetic procedure, and/or in a pathological condition. In general, divalent cations such as calcium ions and/or magnesium ions are included in a physiologically acceptable medium. In some embodiments, divalent cations are balanced with monovalent cations such as sodium and potassium ions at appropriate ratios in order to maintain the homeostasis of skin barrier. The compositions, kits and methods can be used as cosmetics, cosmeceuticals or pharmaceuticals for improving skin condition, and preventing or treating dermatological diseases and skin disorders.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a plot showing calcium concentration in skin sections from the upper layer (Corn1) to the deeper epidermis (Germ) and dermis, before and after treatment with an embodiment of the toner and cream according to the present invention. (Corn: stratum corneum; Gran: stratum granulosum; Spin: stratum spinosum; Germ: stratum germinativum/basale)

FIG. 2 is a plot showing recovery progress of skin damaged by tape-stripping with or without treatment using an embodiment of the toner and cream according to the present invention. Percent recovery is calculated based on TEWL (transepidermal water loss) baseline values/TEWL each day prior to the treatment.

FIG. 3 is plot showing cumulative number of subjects reporting no clinical sign of scaling after number of days using the Dermesis Products. On day 11, 90% subjects reported complete clearing of scaling.

FIG. 4 is a plot showing melanin index before and after use of compositions of the present invention. After 6 hours of use, melanin index decreased and remained normalized for one week.

FIG. 5 is a graph showing facial skin moisture content assessment before and after 6 hours, 1 week, or 2 weeks of use of compositions of the present invention.

FIG. 6 is a graph showing the increase of hydration of the inner arm of subjects immediately after wash. The compositions of the present invention was applied to the volar side of forearm and compared to 3 other moisturizers. The applications were washed from the skin surface after an hour. The moisture content on the site treated with compositions of the present invention was increased 40% compared to baseline, whereas the other moisturizers only provide increase of up to 20%.

FIG. 7 is a graph showing the increase of elasticity of the inner arm of subjects immediately after wash. The compositions of the present invention was applied to the volar side of forearm and compared to 3 other moisturizers. The applications were washed from the skin surface after an hour. The elasticity of the site treated increased, whereas application with the other moisturizers decreased elasticity of the skin.

FIG. 8 are photos showing skin barrier recovery using compositions of the present invention after CO₂ laser resurfacing. The toner and cream of the present invention was applied 3 times a day starting 1 day after CO₂ laser resurfacing. Skin recovery was observed 6 days after CO₂ laser resurfacing procedure.

FIG. 9 are photos showing skin barrier recovery using compositions of the present invention after Ruby Laser Treatment for hyperpigmentation. The toner and cream of the present invention was applied 2 times a day starting 1 day after Ruby Laser Treatment. Skin recovery from inflammation was observed 6 days after treatment and good skin recovery observed after 12 days without common side effects, such as post-inflammatory hyperpigmentation.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention provides innovative compositions and methods for restoring skin barrier function. The invention is based on fundamental understanding of the roles calcium ions play in the regulation of skin barrier function and the intricate balance between monovalent and divalent ions for maintaining the homeostasis of skin barrier.

It is known that the limited amount of calcium in skin compared to sodium, potassium and magnesium enables the skin cells to monitor the high and low level accurately. However, the inventors believe that applying the calcium ions alone to the skin may not only be useless, but can also be harmful, because in monitoring the calcium, the skin also takes into account the presence of other ions. In the case of high calcium only application, the skin will take it as overdosing and trigger negative feedback in response which may cause unpredictable adverse effects. Hence, the inventors believe that the application of divalent ions such as calcium or magnesium ions must be balanced with other components to restore or enhance skin barrier function.

According to the present invention, the compositions and methods are provided for skin care, including prevention or treatment of abnormal skin conditions due to compromised skin barrier function, such as dehydration and inflammation which may be caused by a medical, dermotological or cosmetic procedure, such as chemical peeling, light-activated chemical treatment, laser skin resurfacing, hair removal using chemicals or light energy, vein removal, microdermabrasion, and skin treatment using light energy such as photofacials, which may often result in hyperpigmentation. Other abnormal skin conditions may include pigmented lesions, melasma, cafe au lait and liver spots spots, freckles and lesions observed in Addison's disease, hemochro-matosis, vitiligo, piebaldism, phenylketonuria, and the like.

Without being bound to the particular theory or mechanism of action, the compositions are believed to exert their beneficial effects to the skin through: i) maintaining the balance of major ions in skin, especially between monovalent and divalent cations; ii) restoring the balance of the major ions in skin, especially between monovalent and divalent cations; and iii) restoring and maintaining the growth process of skin cells, which is influenced by the ion balance.

By restoring the balance of major ions in the skin, various beneficial effects can be achieved. As skin receives various challenges from the environment and changes in diet, it tries to adapt by adjusting the quality of skin barrier. When the environment becomes dry, the skin may create stiff barrier to prevent high level of water loss. The stiff barrier can later result in rough and dull skin. Interestingly, skin uses the calcium ions to indirectly monitor the dryness of the environment. If the dryness is exaggerated by other cause, like skin diseases or secondary insults/challenges, the ion balance will be shifted for a long time, triggering the continuous formation of poor quality barrier function. Hence, the restoration of ion balance will induce the regeneration of normal skin barrier layer, which has the optimum barrier function. The formation of a normal skin barrier can also ensure the healthy growth of other cells and components of the skin, such as lymphocytes and keratinocytes, thereby achieving optimum skin conditions.

The inventors also believe that any form of treatment of the skin using light energy within a wide range of wavelength (e.g., from 200 nm/UV, visible light, to 10000 nm/CO₂ laser) will affect the skin barrier integrity. For example, laser treatment uses light energy to impact target sites in deeper locations within the epidermis or dermis. To achieve the required amount of energy on the target site, the initial energy delivered to the skin surface generally is of a higher level and thus affects the skin cells on the surface much more than those at the target site. Loss of skin barrier integrity or impaired skin barrier could result in an array of skin disorders, such as erythema, dehydration, inflammation, hypersensitivity, and hyperpigmentation.

As further demonstrated in the Example section, by restoring the skin barrier function effectively and quickly by using the inventive compositions and methods, post cosmetic procedure inflammation of the skin after laser treatment of the skin or photofacial can be treated, significantly inhibited or prevented.

Recently various methods have been developed for removing superficial skin layers to cause the growth of new skin layers (i.e., commonly described as skin resurfacing techniques) and are used for cosmetic purposes, such as generating tighter, younger looking skin, treating wrinkles, hyperpigmentation, and other skin blemishes or irregularities.

Following the removal of surface skin layers at a particular depth, the body's natural wound-healing response begins to regenerate the epidermis and underlying wounded skin layers. The new skin layer typically cytologically and architecturally resembles younger and more blemish-free normal skin. The range of resurfacing treatments can vary on the depth of the skin removal and wound, for example, superficial exfoliations or peels may extend into the epidermis, medium-depth resurfacing treatments may extend into the papillary dermis, and deep resurfacing treatments may remove tissue to the depth of the reticular dermis.

Techniques for skin layer removal, such as deep resurfacing treatments (e.g., CO₂ laser treatments or ruby laser treatments), extend well into the reticular dermis and may cause significant growth of new skin layers. (See for example, www.almalasers.com). Other laser resurfacing techniques may include Erbium laser resurfacing. Other techniques include mechanical dermabrasion using high-speed abrasive wheels. Microdermabrasion has been developed that uses an air-pressure source to deliver abrasive particles directly against a patient's skin at high-velocities to abrade away skin layers and may be considered superficial resurfacing treatment.

Use of chemical peels may also be used and range from a superficial to a deep resurfacing treatment, depending on the treatment parameters. Superficial exfoliation, peel or abrasion removes some or all of the epidermis and can be suited for treating very light rhytides. Popular superficial chemical peeling agents include α-hydroxy acids, e.g., glycolic acid or other “fruit acids” such as citric and lactic acids; trichloroacetic acid; resorcinol and Jessner's solution. Medium depth peels penetrate to the papillary dermis and typically use 40-50% trichloroacetic acid as the chemical peeling agent. Deep peels penetrate to the reticular dermis and typically use phenol as the chemical peeling agent.

Other cosmetic procedures for skin treatment include radiofrequency therapy. Energy and heat through radiofrequency is applied to skin thereby heating the epidermis and underlying tissue, which tightens the skin, presumably, by heating the underlying collagen and causing it to contract. For example, Thermage (www.thermage.com), uses monopolar capacitive radiofrequency (CRF) to treat skin. Side effects may include redness, swelling, blisters, bumps and surface irregularities.

Plasma energy is a method used for treatment of skin conditions including, but not limited to, facial and non-facial rhytides, superficial skin lesions, actinic keratosis, seborrhoeic keratosis, and viral papillomata. Plasma energy skin therapy is available, for example, through Rhytec (www.rhytec.com). Plasma is a gas in which atoms have been ionized or stripped of electrons. The thermal energy from the plasma is thought to be absorbed by the skin, creating growth conditions for new collagen and skin regeneration for a natural, more youthful appearance. By delivering plasma energy deep into the dermis, new epidermis emerges as the old surface epidermis begins to shed.

Photofacials are a series of full face, gentle pulsed light treatments that may improve the appearance of sun damaged and aged skin, as well as reduce facial and neck redness and flushing.

However, these procedures have drawbacks. The epidermis is typically negatively affected by these cosmetic procedures. In many cases, inflammation occurs as the skin barrier is damaged, and the skin invokes defense reactions. Uncontrolled epidermal reactions may lead to adverse effects, such as erythema, dry skin, inflammation, and/or post-inflammatory hyperpigmentation (PIH). Another significant disadvantage relates to the post-treatment recovery period. For example, it may require up to several weeks or even months to fully recover and to allow the skin the form a new epidermal layer. During a period ranging from a few weeks to several weeks after a deep resurfacing treatment, the new skin is usually bright pink or red. The skin may also be more sensitive.

The compositions of the present invention can help alleviate the impact of these cosmetic procedures and decrease the time for recovery of the skin barrier after these procedures by normalizing melanin production to prevent hyperpigmentation, and restoring and maintaining skin moisture content and elasticity. Skin layer removal, hair removal, vein removal and lightening, and applications of energy on the skin has typically has an impact on the condition of skin barrier function and the present invention may lessen the negative impacts of cosmetic procedures on skin barrier function. The method according to claim 26, wherein the skin barrier function of the subject is restored at least 2-15 days faster than the time required for restoration of the skin barrier function of another subject who has gone through the same cosmetic procedure but without being treated with the composition.

According to the present invention, the skin barrier function of the subject may be restored at least 5-12 days faster than the time required for restoration of the skin barrier function of another subject who has gone through the same procedure but without being treated with the composition.

Also according to the present invention, the inflammation of the skin of the subject is reduced at least 50% within a time period at least 2-15 days shorter than the time required for at least 50% reduction of inflammation of the skin of another subject who has gone through the same procedure but without being treated with the composition.

Also according to the present invention, the composition may be administered topically to the subject prior to the procedure, e.g., at least 1-10 days prior to the procedure.

Also according to the present invention, the composition may be administered topically to the subject at least 1-60 minutes after the procedure. In addition, the composition may be administered topically to the subject prior to, concurrently, or post the procedure.

In one aspect of the invention, a composition for skin care is provided. In one embodiment the composition comprises about 0.01-8% w/w divalent calcium ion and/or divalent magnesium ion based on the total weight of the composition in a physiologically acceptable medium. Optionally, the ratio of divalent calcium ion to divalent magnesium ion ranges from 5:1 to 1:5, from 3:1 to 1:3, from 2:1 to 1:2, or from 3:2 to 2:3. The physiologically acceptable medium is preferably a cosmetically or pharmaceutically acceptable carrier. Preferably, calcium ions are provided in the form of calcium chloride; and magnesium ions are provided in the form of magnesium chloride.

In another embodiment, the composition comprises divalent calcium ion and monovalent ion at a ratio of 15:1 to 1:20 in a physiologically acceptable medium, wherein the amount of the divalent calcium ion is about 0.01-8% by weight relative to the total weight of the composition. Optionally, the ration of divalent calcium ion to monovalent ion ranges from 8:1 to 1:10, from 6:1 to 1:3, from 4:1 to 1:5, from 2:1 to 1:3, or from 1:1 to 1:2. Preferably, the monovalent ions are sodium or potassium ions in a form of sodium chloride, potassium chloride or potassium bromide.

According these embodiments, in the compositions divalent cations (e.g., calcium or magnesium cations) are combined with monovalent cations (e.g., sodium or potassium cations) at a proper ratio in order to maintain the balance of ions in skin barrier and avoid triggering negative back in response. The divalent cations can also be combined with other major skin components as cholesterol, fatty acids and amino acids or their equivalents.

In a particular embodiment, the divalent calcium or magnesium ion are preferably combined with monovalent sodium and potassium ion in an aqueous liquid medium. The aqueous liquid medium constitutes an aqueous phase, which may be the continuous phase of the composition.

The aqueous phase may consist essentially of water; it may also comprise a mixture of water and of water-miscible solvent (miscibility in water of greater than 50% by weight at 25° C.), for instance lower monoalcohols containing from 1 to 5 carbon atoms such as ethanol or isopropanol, glycols containing from 2 to 8 carbon atoms, such as propylene glycol, ethylene glycol, 1,3-butylene glycol or dipropylene glycol, C₃-C₄ ketones and C₂-C₄ aldehydes, and glycerin.

The aqueous phase (water and optionally the water-miscible organic solvent) may be present in a content ranging from 1% to 98% by weight, relative to the total weight of the composition, optionally from 3% to 96%, from 40% to 95%, from 50% to 90%, from 60% to 90%, or from 70% to 85%.

In the aqueous liquid formulation, the amount of divalent calcium ion is preferably about 0.1-8%, optionally about 0.5-5%, or optionally about 1-3%. The divalent calcium ion is preferably provided by adding CaCl₂ to the aqueous phase.

Such an aqueous formulation can be used as skin toner, moisturizer or humectant to promote skin barrier repair and restore normal skin barrier function to skin exposed to or damaged by environmental elements or in pathological conditions.

Optionally, the composition of the present invention is in a form of emulsion or cream formulation. It can contain emulsifying surfactants, present in particular in a proportion ranging from 2% to 30% by weight relative to the total weight of the composition, and better still from 5% to 15%. These surfactants may be chosen from anionic and nonionic surfactants. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of said reference, for the anionic and nonionic surfactants.

The surfactants preferably used in the composition according to the invention are chosen from: nonionic surfactants: fatty acids, fatty alcohols, polyethoxylated or polyglycerolated fatty alcohols such as polyethoxylated stearyl or cetylstearyl alcohol, fatty acid esters of sucrose, alkylglucose esters, in particular polyoxyethylenated fatty esters of C₁-C₆ alkyl glucose, and mixtures thereof; anionic surfactants: C₁₆-C₃₀ fatty acids neutralized with amines, aqueous ammonia or alkaline salts, and mixtures thereof. Surfactants which make it possible to obtain an oil-in-water or wax-in-water emulsion are preferably used.

In the cream formulation, the amount of divalent calcium ion is preferably about 0.01-8%, optionally about 0.05-0.5%, or optionally about 0.1-0.3%. The divalent calcium ion is preferably provided by adding CaCl₂ to the emulsion.

Optionally, the composition of the present invention is in a form of aqueous gel or hydrogel formulation. The hydrogel formulation comprises a thickening agent to thicken the liquid solution. Examples of the thickening agents include, but are not limited to, carbomers, cellulose base materials, gums, algin, agar, pectins, carrageenan, gelatin, mineral or modified mineral thickeners, polyethylene glycol and polyalcohols, polyacrylamide and other polymeric thickeners. The thickening agents which give the stability and optimal flow characteristics of the composition are preferably used.

In the hydrogel formulation, the amount of divalent calcium ion is preferably about 0.01-8%, optionally about 0.05-0.5%, or optionally about 0.1-0.3%. The divalent calcium ion is preferably provided by adding CaCl₂ to the formulation.

The compositions according to the invention may further comprise an effective amount of a physiologically acceptable antioxidant selected from the group consisting of butylated p-cresol, butylated hydroquinone monomethyl ether, and a tocopherol. The antioxidant can be present in amounts of 0.005-5% by weight of the total composition.

The compositions according to the invention may further comprise natural or modified amino acid, such as arginine, cystine, glutamine, histidine, isoleusine, leucine, lysine, methionine, phenylalanine, threonine, tryptophan, tyrosine, and valine. The amino acid can be present in amounts of from 0.002-6% by weight of the total composition.

The compositions according to the invention may further comprise natural or modified sterol compound such as cholesterol and plant sterol (also called phytosterol) such as stigmasterol, campesterol, β-sitosterol, chalinosterol, clionasterol, brassicasterol, α-spinasterol, dancosterol, desmosterol, and poriferasterol. The amino acid can be present in amounts of from 0.001-5% by weight of the total composition.

The compositions according to the invention may further comprise natural or modified collagen, silk protein or soy protein. The protein can be present in amounts of from 0.01-10% by weight of the total composition.

The compositions according to the invention are preferably formulated for topical application to keratin materials such as the skin, the hair, the eyelashes or the nails. They may be in any presentation form normally used for this type of application, especially in the form of an aqueous or oily solution, an oil-in-water or water-in-oil emulsion, a silicone emulsion, a microemulsion or nanoemulsion, an aqueous or oily gel or a liquid, pasty or solid anhydrous product.

The inventive compositions may be more or less fluid and may have the appearance of a white or colored cream, an ointment, a milk, a lotion, a serum, a paste, a mousse or a gel. They may optionally be topically applied onto the skin in the form of an aerosol, a patch or a powder. They may also be in solid form, for example, in the form of a stick. They may be used as care products and/or as makeup products for the skin. Alternatively, they may be formulated as shampoos or conditioners.

In known fashion, the compositions of the invention may also contain additives and adjuvants that are common in cosmetics, such as hydrophilic or lipophilic gelling agents, preservatives, antioxidants, solvents, fragrances, fillers, pigments, odor absorbers and dyestuffs. The amounts of these various additives and adjuvants are those conventionally employed in the field under consideration, and range, for example, from 0.01% to 20% of the total weight of the composition. Depending on their nature, these additives and adjuvants may be introduced into the fatty phase or into the aqueous phase.

When the composition according to the invention is an emulsion, the proportion of the fatty phase advantageously ranges from 2% to 80% by weight and preferably from 5% to 50% by weight relative to the total weight of the composition. The fatty substances, emulsifiers and co-emulsifiers included in the composition in emulsion form are selected from among those conventionally formulated in the field under consideration. The emulsifier and co-emulsifier are preferably present in the composition in a proportion ranging from 0.3% to 30% by weight and preferably from 0.5% to 20% by weight relative to the total weight of the composition.

Exemplary fatty substances according to the invention include the oils and especially mineral oils (liquid petroleum jelly), oils of plant origin (avocado oil, evening primrose oil, safflower oil, soybean oil, wheat germ oil, apricot kernel oil), oils of animal origin (lanolin), synthetic oils (perhydrosqualene), silicone oils (cyclomethicone) and fluoro oils (perfluoro polyethers). Fatty alcohols such as cetyl alcohol, fatty acids, waxes and gums and in particular silicone gums are also representative fatty substances.

Exemplary emulsifiers and co-emulsifiers according to the invention include fatty acid esters of polyethylene glycol, such as PEG-100 stearate, PEG-50 stearate and PEG-40 stearate; fatty acid esters of polyols, such as glyceryl stearate, sorbitan tristearate and oxyethylenated sorbitan stearates commercially available under the trademark Tween™20 or Tween™60, for example; and mixtures thereof.

And exemplary hydrophilic gelling agents include in particular, carboxyvinyl polymers (carbomer), acrylic copolymers such as acrylate/alkyl acrylate copolymers, polyacrylamides, polysaccharides, natural gums and clays. Exemplary lipophilic gelling agents include, in particular, modified clays, for example bentones, metal salts of fatty acids and hydrophobic silica.

The present invention also features a cosmetic regime/regimen for skin care by topical application thereon of a composition containing, formulated into a physiologically acceptable medium (vehicle, diluent or carrier), 0.01-15% w/w divalent calcium ion, either alone or in combination with at least one other compound as described above.

This invention relates more particularly to a cosmetic regime or regimen for treating the adverse signs of aging of the skin and/or a dull complexion and/or skin or hair pigmentation disorders and/or skin dryness and/or hyperseborrhoea and/or hyperseborrhoea-related imperfections and/or sensitive skin and/or dandruff and/or natural hair loss and/or baldness, comprising the topical application onto the skin or the hair, for such period of time as required to elicit the desired cosmetic/therapeutic response, of a composition containing, formulated into a physiologically acceptable medium, 0.01-15% w/w divalent calcium ion, either alone or in combination with at least one other compound as described above.

By the expression “signs of aging of the skin” are intended wrinkles and fine lines, loss of firmness and/or elasticity of the skin, cutaneous atrophy, a more irregular skin grain with presence of dilated pores, loss of radiance of the skin and/or pigmentary marks.

By the expression “sensitive skin” is intended skin that has been characterized in EP-0,680,749 B1, hereby incorporated by reference. It has thus been shown that the symptoms associated with sensitive skin included more or less painful sensations experienced in an area of skin, such as stinging, tingling, itching or pruritus, burning, redness, hotness, discomfort, tautness, etc. These symptoms may be manifested in response to various factors such as, inter alia, sweat, friction, the emotions, foods, the wind, shaving, soap, surfactants, hard water with a high calcium concentration, temperature variations or wool.

Thus invention also features cosmetic compositions containing, in a physiologically acceptable medium therefor (vehicle, diluent or carrier), 0.01-15% w/w divalent calcium ion, and at least one other active agent compound selected from among: a desquamating agent, a moisturizer, a depigmenting or propigmenting agent, an anti-glycation agent, an NO-synthase inhibitor, a 5α-reductase inhibitor, a lysyl and/or prolyl hydroxylase inhibitor, an agent for stimulating the synthesis of dermal or epidermal macromolecules and/or for preventing their degradation, an agent for stimulating the proliferation of fibroblasts and keratinocytes and/or keratinocyte differentiation, a muscle relaxant, a compound for reducing irritation, an antimicrobial agent, a tensioning agent, an anti-pollution agent or a free-radical scavenger.

The present invention also features cosmetic compositions containing, in a physiologically acceptable medium, 0.01-15% w/w divalent calcium ion, and at least one UV-screening agent selected from among certain UVA and/or UVB screening agents and/or at least one optionally coated inorganic pigment.

The compositions according to the invention are well suited for topical application onto keratin substrates/materials such as the skin, keratin fibers (head hairs and eyelashes) and the nails.

By the expression “physiologically acceptable medium” is intended a medium that is compatible with the skin and/or its integuments.

Various compounds that may be formulated into the compositions according to the invention will now be more fully described.

1. Desquamating Agents and Moisturizers:

By the term “desquamating agent” is intended any compound capable of acting:

(a) either directly on desquamation by promoting exfoliation, such as α-hydroxy acids, in particular salicylic acid and derivatives thereof (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; hydroxystilbenes including, in particular, resveratrol;

(b) or on the enzymes involved in the desquamation or degradation of corneodesmosomes, glycosidases, stratum corneum chymotryptic enzyme (SCCE), or even other proteases (trypsin, chymotrypsin-like). Exemplary agents for chelating mineral salts are EDTA; N-acyl-N,N′,N′-ethylenediaminetriacetic acid; amino-sulfonic compounds and in particular (N-2-hydroxyethylpiperazine-N-2-ethane)sulfonic acid HEPES); 2-oxothiazolidine-4-carboxylic acid (procysteine); .alpha.-amino acid derivatives of the type such as glycine (as described in EP-0,852,949); honey; sugar derivatives such as O-octanoyl-6-D-maltose and N-acetylglucosamine.

By the term “moisturizer” is intended:

(a) either a compound acting on the barrier function, in order to maintain the moisturization of the stratum corneum, or an occlusive compound. Exemplary are the ceramides, sphingoid-based compounds, lecithins, glycosphingolipids, phospholipids, cholesterol and derivatives thereof, phytosterols (stigmasterol, □-sitosterol or carnpesterol), essential fatty acids, 1,2-diacylglycerol, 4-chromanone, pentacyclic triterpenes, petroleum jelly and lanolin;

(b) or a compound that directly increases the water content of the stratum corneum, such as threalose and derivatives thereof, hyaluronic acid and derivatives thereof, glycerol, pentanediol, pidolates, amino acids (for examples serine, proline, glutamates, arginine), xylitol, urea, creatine, glucosamines, lactic acid, lactates, polyglyceryl acrylate, ectoin and derivatives thereof, chitosan, sugars, oligosaccharides and polysaccharides, cyclic carbonates, polyaspartate and derivates thereof, pyrrolidone-carboxylic acid and derivatives thereof, N-lauroyl-pyrrolidonecarboxylic acid, N-lauroyl-lysine and N-.alpha.-benzoyl-L-arginine;

(c) or a compound that activates the sebaceous glands, such as steroid derivatives (including DHEA) and vitamin D and derivatives thereof.

These compounds advantageously constitute from 0.001% to 30% and preferably from 0.01% to 20% of the total weight of the composition according to the invention.

The compositions according to the present invention comprising the desquamating agents and moisturizers indicated above are well suited for preventing or treating skin dryness and especially xerosis.

2. Depigmenting or Propigmenting Agents:

Exemplary depigmenting agents that may be formulated into the compositions according to the present invention comprise the following compounds: kojic acid; ellagic acid; arbutin and derivatives thereof such as those described in EP-895,779 and EP-524,109; hydroquinone; aminophenol derivatives such as those described in WO-99/10318 and WO-99/32077, and in particular N-cholesteryloxycarbonyl-para-aminophenol and N-ethyloxycarbonyl-para-aminophenol; iminophenol derivatives, in particular those described in WO-99/22707; L-2-oxothiazolidine-4-carboxylic acid or procysteine, and also its salts and esters; ascorbic acid and derivatives thereof, especially ascorbyl glucoside; and plant extracts, in particular extracts of liquorice, of mulberry and of skullcap, this list not intended to be limiting.

Propigmenting agents that are exemplary include the extract of burnet (Sanguisorba officinalis) marketed by Maruzen, and extracts of chrysanthemum (Chrysanthemum morifolium).

The compositions according to the present invention comprising the depigmenting agents indicated above are well suited for preventing or treating hyperpigmentation, in particular pigmentary marks related to aging of the skin.

The compositions containing the propigmenting agents indicated above are well suited for treating baldness.

3. Anti-Glycation Agents:

By the term “anti-glycation agent” in intended a compound for preventing and/or reducing the glycation of skin proteins, in particular of dermal proteins such as collagen.

Exemplary anti-glycation agents are plant extracts of the Ericacea family, such as an extract of blueberry (Vaccinium angustifolium); ertothioneine and derivatives thereof; and hydroxystilbenes and derivatives thereof, such as resveratrol and 3,3′,5,5′-tetrahydroxystilbene. These anti-glycation agents are described in FR-99/16166, FR-00/08158, FR-99/09267 and FR-99/16168, respectively. Resveratrol is particularly preferred for formulation into the compositions of the invention.

The compositions of the invention comprising an anti-glycation agent as defined above are well suited to prevent or treat the signs of aging of the skin, in particular to prevent or treat the loss of tonicity and/or elasticity of the skin.

4. NO-Synthase Inhibitors:

Exemplary NO-synthase inhibitors that are suitable for formulation into the compositions of the present invention especially comprise a plant extract of the species Vitis vinifera which is marketed by Euromed as Leucocyanidines de raisins extra, or by Indena under the trademark Leucoselect®, or also by Hansen as Extrait de marc de raisin; a plant extract of the species Olea europaea which is preferably obtained from olive tree leaves and is marketed by Vinyals in the form of a dry extract, or by Biologia & Technologia under the trademark Eurol BT; and a plant extract of the species Gingko biloba which is preferably a dry aqueous extract of this plant marketed by Beaufour as Gingko biloba extrait standard.

The compositions according to the invention comprising an NO-synthase inhibitor as defined above are well suited to prevent or treat the signs of aging of the skin and/or sensitive skin.

5. 5α-Reductase Inhibitors:

When the compositions according to the invention comprise a 5α-reductase inhibitor, such inhibitor is advantageously selected from among:

retinoids, and in particular retinol;

sulfur and sulfur derivatives;

zinc salts such as zinc lactate, gluconate, pidolate, carboxylate, salicylate and/or cysteate;

selenium chloride;

vitamin B6 or pyridoxine;

mixture of capryloyl glycine, sarcosine and extract of Cinnamomum zeylanicum marketed by Seppic under the trademark Sepicontrol A5®;

an extract of Laminaria saccharina marketed by SECMA under the trademark Phlorogine®;

an extract of Spiraea ulmaria marketed by Silab under the trademark Sebonormine™;

plant extracts from the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all marketed, for example, by Maruzen;

an extract of Serenoa repens marketed by Euromed; plant extracts of the genus Silybum;

plant extracts containing sapogenins and in particular extracts of diosgenin-rich or hecogenin-rich Dioscorea plants; and extracts of Eugenia caryophyllata containing eugenol or eugenyl glucoside.

The 5α-reductase inhibitor advantageously constitutes, for example, from 0.001% to 10% and preferably from 0.01% to 5% of the total weight of the composition according to the invention. When this composition contains such a compound, it is particularly suitable for preventing or treating seborrhoea and/or hirsutism and/or androgen-dependent alopecia.

6. Lysyl and/or Prolyl Hydroxylase Inhibitors:

Preferred examples of lysyl and/or propyl hydroxylase inhibitors that may be formulated into the compositions according to the present invention are 2,4-diaminopyrimidine 3-oxide or 2,4-DPO described in WO-96/09048 and 2,4-diamino-6-piperidinopyrimidine 3-oxide or “Minoxidil” described in U.S. Pat. Nos. 4,139,619 and 4,596,812.

These compounds are advantageously present, for example, in the compositions of the invention in a proportion of from 0.001% to 5% by weight and preferably in a proportion of from 0.01% to 5% by weight relative to the total weight of the composition.

7. Agents for Stimulating the Synthesis of Dermal or Epidermal Macromolecules and/or for Preventing Their Degradation:

Among the active agents for stimulating dermal macromolecules, exemplary are those that act:

(a) either on collagen synthesis, such as extracts of Centella asiatica; asiaticosides and derivatives thereof; ascorbic acid or vitamin C and derivatives thereof; synthetic peptides such as lamin, biopeptide CL or the palmitoyloligopeptide marketed by Sederma; peptides extracted from plants, such as the soybean hydrolysate marketed by Coletica under the trademark Phytokine®; plant hormones such as auxins and cinnamic acid and derivatives thereof, as described in the European patent application published under No. 0,925,779;

(b) or on elastin synthesis, such as the extract of Saccharomyces cerivisiae marketed by LSN under the trademark Cytovitin™; and the extract of the alga Macrocystis pyrifera marketed by SECMA under the trademark Kelpadelie®;

(c) or on glycosaminoglycan synthesis, such as the product of fermentation of milk with Lactobacillus vulgaris, marketed by Brooks under the trademark Biomin Yogourt®; the extract of the brown alga Padina pavonica marketed by Alban Muller under the trademark HSP®; and the extract of Saccharomyces cerevisiae available from Silab under the trademark Firmalift® or from LSN under the trademark Cytovitin®;

(d) or on fibronectin synthesis, such as the extract of the zooplankton Salina marketed by Seporga under the trademark GP4G®; the yeast extract available from Alban Muller under the trademark Drieline®; and the palmitoyl pentapeptide marketed by Sederma under the trademark Matrixil®;

(e) or on metalloprotease (MMP) inhibition, such as, more particularly, MMP 1, 2, 3 or 9: exemplary are the retinoids and derivatives, isoflavonoids, oligopeptides and lipopeptides, lipoamino acids, the malt extract marketed by Coletica under the trademark Collalift®; extracts of blueberry or of rosemary; carotenoids including, in particular, lycopene; isoflavones, their derivatives or plant extracts containing them, in particular extracts of soybean (marketed, for example, by Ichimaru Pharcos under the trademark Flavosterone SB®), of red clover, of flax, of kakkon, of sage or extracts of sage (as described in French patent application No. 00/10203);

(f) or on the inhibition of serine proteases such as leukocyte elastase or cathepsin G: exemplary are the peptide extract of Leguminosa seeds (Pisum sativum) marketed by LSN under the trademark Parelastyl®, and heparinoids and pseudodipeptides.

Among the active agents that stimulate epidermal macromolecules, such as fillagrin and keratins, especially representative are the extract of lupin marketed by Silab under the trademark Structurine®; the extract of beech Fagus sylvatica buds marketed by Gattefosse under the trademark Gatuline®, and the extract of the zooplankton Salina marketed by Seporga under the trademark GP4G®.

The compositions according to the invention containing one or more of the above compounds are particularly suitable for preventing or treating the signs of aging of the skin, in particular loss of firmness and/or elasticity of the skin.

8. Agents for Stimulating the Proliferation of Fibroblasts or Keratinocytes and/or Keratinocyte Differentiation:

Exemplary agents for stimulating the proliferation of fibroblasts that may be formulated into the compositions of the invention include plant proteins or polypeptides, extracts, especially of soybean (for example an extract of soybean marketed by LSN under the trademark Eleseryl SH-VEG8® or marketed by Silab under the trademark Raffermine®); and plant hormones such as giberrellins and cytokinins.

The agents for stimulating keratinocyte proliferation that may be formulated into the compositions according to the invention especially comprise retinoids such as retinol and its esters, including retinyl palmitate; retinoid acids and derivates thereof, extracts of nut cakes marketed by Gattefosse; and extracts of Solanum tuberosum marketed by Sederma.

The agents for stimulating keratinocyte differentiation comprise, for example, minerals such as calcium; the extract of lupin marketed by Silab under the trademark Photopreventine®; sodium beta-sitosteryl sulfate marketed by Seporga under the trademark Phytocohesine®; and the extract of corn marketed by Solabia under the trademark Phytovityl®.

The compositions according to the invention comprising these compounds are preferably used for preventing or treating the signs of aging of the skin.

9. Muscle Relaxants:

The muscle relaxants that may be included in the compositions according to the invention comprise calcium inhibitors such as alverine and its salts, chlorine-channel openers such as diazepam, and catecholamine and acetylcholine inhibitors, such as the hexapeptide argireline R marketed by Ilipotec.

The compositions of the invention comprising these compounds are used for preventing or treating the signs of aging of the skin and in particular wrinkles.

10. Antimicrobial Agents:

Exemplary antimicrobial agents that may be formulated into the compositions according to the invention include 2,4,4′-trichloro-2′-hydroxydiphenyl ether (or triclosan), 3,4,4′-trichloro-banilide, phenoxyethanol, phenoxypropanol, phenoxy-isopropanol, hexamidine isethionate, metronidazole and its salts, micronazole and its salts, itraconazole, terconazole, econazole, ketoconazole, saperconazole, fluconazole, clotrimazole, butoconazole, oxiconazole, sulphaconazole, sulconazole, terbinafine, ciclopirox, ciclopiroxolamine, undecylenic acid and its salts, benzoyl peroxide, 3-hydroxybenzoic acid, 4-hydroxy-benzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerine, octanoylglycine, caprylyl glycol, 10-hydroxy-2-decanoic acid, dichlorophenylimidazole dioxolane and its derivatives described in WO-93/18743, framesol and phytosphingosines, and mixtures thereof.

The preferred antimicrobial agents are triclosan, phenoxyethanol, octoxyglycerine, octanoyl-glycine, 10-hydroxy-2-decanoic acid, caprylyl glycol, farnesol and azelaic acid.

By way of example, the antimicrobial agents may be formulated into the compositions of the invention in amounts advantageously representing from 0.1% to 20% and preferably from 0.1% to 10% of the total weight of the composition.

11. Tensioning Agents:

By the term “tensioning agent” is intended a compound capable of exerting tension on the skin, the effect of which is to temporarily fade out irregularities on the skin's surface, such as wrinkles and fine lines.

Among the tensioning agents that may be formulated into the compositions of the present invention, especially representative are:

(1) polyurethane latices or acrylic-silicone latices, in particular those described in EP-1,038,519, such as a propylthio(polymethylacrylate), propylthio(polymethyl methacrylate) or propylthio(polymethacrylic acid) grafted polydimethyl-siloxane, or alternatively a propylthio(polyisobutyl methacrylate) and propylthio(polymethacrylic acid) grafted polydimethylsiloxane. Such grafted silicone polymers are marketed by 3M under the trademark VS 80, VS 70 or LO21.

(2) soybean or wheat plant proteins, and/or

(3) sodium magnesium silicates (Laponites).

The compositions according to the invention comprising the above tensioning agents are well suited for treating the signs of aging of the skin, in particular wrinkles and fine lines.

12. Immunomodulatory Agents:

By the term “immunomodulatory agent” is intended any compound capable of either stimulate or suppress the immune response of the body, such as steroids, corticoids, azathioprine, mercaptopurines, methotrexate, mycophenolic acid and derivates thereof, leflunomide and derivates thereof, or cyclophosphamides.

13. Anti-Pollution Agents or Free-Radical Scavengers:

By the term “anti-pollution agent” is intended any compound capable of trapping ozone, monocyclic or polycyclic aromatic compounds such as benzopyrene and/or heavy metals such as cobalt, mercury, cadmium and/or nickel. By the term “free-radical scavenger” is intended any compound capable of trapping free radicals.

Exemplary ozone-trapping agents that may be formulated into the compositions according to the invention are, in particular, vitamin C and derivatives thereof, including ascorbyl glucoside; phenols and polyphenols, in particular tannins, ellagic acid and tannic acid; epigallocatechin and natural extracts containing same; extracts of olive tree leaf; extracts of tea, in particular of green tea; anthocyans; extracts of rosemary; phenol acids, in particular chlorogenic acid; stilbenes, in particular resveratrol; sulfur-containing amino acid derivatives, in particular S-carboxymethylcysteine; ergothioneine; N-acetylcysteine; chelating agents, for instance N,N′-bis(3,4,5-trimethoxybenzyl)ethylenediamine or one of its salts, metal complexes or esters; carotenoids such as crocetin; and various starting materials, for instance the mixture of arginine, histidine ribonucleate, mannitol, adenosine triphosphate, pyridoxine, phenyl-alanine, tyrosine and hydrolysed RNA, marketed by Laboratoires Serobiologiques under the trademark CPP LS 2633-12F®, the water-soluble fraction of corn marketed by Solabia under the trademark Phytovityl®, the mixture of extract of fumitory and of extract of lemon marketed under the trademark Unicotrozon C-49® by Induchem, and the mixture of extracts of ginseng, of apple, of peach, of wheat and of barley, marketed by Provital under the trademark Pronalen Bioprotect®.

Exemplary agents for trapping out monocyclic or polycyclic aromatic compounds according to the invention are, in particular, tannins such as ellagic acid; indole derivatives, in particular 3-indolecarbinol; extracts of tea, in particular of green tea, extracts of water hyacinth or Eichhornia crassipes; and the water-soluble fraction of corn marketed by Solabia under the trademark Phytovityl®.

Finally, exemplary heavy-metal-trapping agents that may be formulated into the compositions according to the invention include, in particular, chelating agents such as EDTA, the pentasodium salt of ethylenediamine tetramethylenephosphonic acid, and N,N′-bis(3,4,5-trimethoxybenzyl)ethylenediamine or one of the salts, metal complexes or esters thereof; phytic acid; chitosan derivatives; extracts of tea, in particular of green tea; tannins such as ellagic acid; sulfur-containing amino acids such as cysteine; extracts of water hyacinth (Eichhornia crassipes); and the water-soluble fraction of corn marketed by Solabia under the trademark Phytovityl®.

The free-radical scavengers that may be included in the compositions according to the invention comprise, other than certain anti-pollution agents indicated above, vitamin E and derivatives thereof such as tocopheryl acetate; bioflavonoids; coenzyme Q10 or ubiquinone; certain enzymes, for example, catalase, superoxide dismutase, lactoperoxidase, glutathione peroxidase and quinone reductases; glutathione; benzylidenecamphor; benzylcyclanones; substituted napthalenones; pidolates; phytanetriol; gamma-oryzanol; lignans; and melatonin.

The compositions of the invention comprising the anti-pollution agents and/or free-radical scavengers indicated above are well suited for preventing or treating the signs of aging of the skin, in particular wrinkles, and loss of firmness and elasticity of the skin and dehydration. As a variant, same are useful for preventing or treating a dull complexion.

14. UVA and/or UVB Screening Agents and Optionally Coated Inorganic Pigments:

The compositions according to the invention may contain one or more UV-screening agents capable of screening out UVA and/or UVB radiation.

Exemplary compounds for screening out UVA radiation include, especially:

(a) benzophenone derivatives, for example:

2,4-dihydroxybenzophenone (benzophenone-1);

2,2′,4,4′-tetrahydroxybenzophenone (benzo-phenone-2);

2-hydroxy-4-methoxybenzophenone (benzo-phenone-3), available from BASF under the trademark Uvinul M40;

2-hydroxy-4-methoxybenzophenone-5-sulfonic acid (benzophenone-4) and also its sulfonate form (benzophenone-5), available from BASF under the trademark Uvinul MS40;

2,2′-dihydroxy-4,4′-dimethoxybenzophenone (benzophenone-6);

5-chloro-2-hydroxybenzophenone (benzophenone-7);

2,2′-dihydroxy-4-methoxybenzophenone (benzo-phenone-8);

the disodium salt of 2,2′-dihydroxy-4,4′-dimethoxybenzophenone-5,5′-disulfonic acid (benzophenone-9);

2-hydroxy-4-methoxy-4′-methylbenzophenone (benzophenone-10);

benzophenone-11;

2-hydroxy-4-(octyloxy)benzophenone (benzo-phenone-12);

benzophenones 3 and 5 being preferred;

(b) triazine derivatives, and in particular 2,4-bis{[4-(2-ethylhexyloxy)-2-hydroxy]phenyl}-6-(4-methoxy-phenyl)-1,3,5-triazine available from Ciba Geigy under the trademark Tinosorb 5 and 2,2′-methylenebis[6-(2H-benzotriazol-2-yl)-4-(1,1,3,3-tetramethylbutyl)phenol] available from Ciba Geigy under the trademark Tinosorb M;

(c) benzene-1,4-bis(3-methylidene-10-camphorsulfonic acid), optionally in partially or totally neutralized form, and

(d) mixtures thereof.

Exemplary compounds for screening out UVB radiation include:

(a) salicylic acid derivatives, in particular homomethyl salicylate and octyl salicylate;

(b) cinnamic acid derivatives, in particular 2-ethylhexyl p-methoxycinnamate, available from Givaudan under the trademark Parsol MCX;

(c) liquid .beta.,.beta.′-diphenylacrylate derivatives, in particular 2-ethylhexyl .alpha.-cyano-.alpha.,.beta.′-diphenylacrylate, or octocrylene, available from BASF under the trademark Uvinul N539;

(d) p-aminobenzoic acid derivatives;

(e) 4-methylbenzylidenecamphor available from Merck under the trademark Eusolex 6300;

(f) 2-phenylbenzimidazole-5-sulfonic acid marketed under the trademark “Eusolex 232” by Merck;

(g) 1,3,5-triazine derivatives, in particular: and

2,4,6-tris[p-(2′-ethylhexyl-1′-oxycarbonyl)anilino]-1,3,5-triazine, available from BASF under the trademark Uvinul T150.

Exemplary compounds for screening out UVA and UVB radiation are, in particular:

plant extracts, in particular of rosemary (rosmarinic acid) and of the genus Leontopodium, in particular a plant species Leontopodium alpinum or Leontopodium stracheyi; and benzotriazole silicone, described in FR-A-2,642,968.

Exemplary optionally coated inorganic pigments include nanopigments of titanium dioxide, of iron oxide, of zinc oxide, of zirconium oxide or of cerium oxide optionally coated with alumina and/or with aluminum stearate.

15. Compounds of Neurogenic Origin for Reducing Irritation:

Exemplary compounds of neurogenic origin for reducing irritation include:

(a) substance P antagonists and in particular those described in EP-0,680,749, extracts of at least one non-photosynthetic filamentous bacterium, particularly strains of Vitreoscilla filiformis described in EP-0,761,204, the spring waters described in EP-0,764,440, extracts of at least one plant of the Rosacea family, particularly of the species Rosa gallica described in the European patent application published under No. 0,906,752 and the alkaline earth metals described in the European patent applications published under Nos. 0,737,471 and 0,770,302;

(b) CGRP antagonists, in particular those described in EP-0,765,668 and especially Iridacea extracts, particularly of the species Iris pallida;

(c) NO-synthase inhibitors;

(d) bradykinin antagonists and in particular those described in the European patent application published under No. 0,909,556;

(e) cytokine antagonists;

(f) histamine antagonists;

(g) antagonists of interleukin 1 and/or of tumor necrosis factor of a type (TNFα) and in particular those described in the European patent applications published under Nos. 0,892,642 and 0,764,444, particularly peptide Modulene, the tripeptide Lysine-Proline-Valine (KPV) and an extract of at least one plant from the Labiae family, particularly of the species Rosmarinus officinalis;

(h) sodium-channel blockers preferably selected from among: Amiloride, Quinidine, Quinidine sulfate, Apamine, Cyproheptadine, Loperamide and N-acetylprocainamide

(i) potassium-channel openers, especially Minoxidil and derivatives thereof.

The composition should also comprise a vehicle to enable the active ingredient to be conveyed to the skin in an appropriate dilution. The composition may be in a form of liquid, suspension, emulsion, lotion or cream.

The selection of a vehicle for the active ingredient(s) in compositions of the invention presents a wide range of possibilities depending on the required product from of the composition. Suitable vehicles can be classified as described hereinafter.

It should be explained that vehicles are substances which can act as diluents, dispersants, or solvents for the active ingredients and which therefore ensure that they can be applied to and distributed evenly over the skin at an appropriate concentration; the vehicle is preferably one which can aid penetration of the active ingredient into the skin, thus ensuring that the effectiveness of the active ingredient is prolonged because of improved properties. Compositions according to this invention can include water is a vehicle, and/or at least one cosmetically acceptable vehicle other than water.

Vehicles other than water that can be used in compositions according to the invention can include solids or liquids such as emollients, propellants, solvents, humectants, thickeners and powders. Examples of each of these types of vehicles, which can be used singly or as mixtures of one or more carriers, are as follows:

Emollients, such as stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polyethylene glycol, triethylene glycol, lanolin, castor oil, acetylated lanolin alcohols, petroleum, mineral oil, butyl myristate, isostearic acid, palmitic acid, isopropyl linoleate, lauryl lactate, myristyl lactate, decyl oleate, myristyl, myristate;

Propellants, such as trichlorofluoromethane, dichlorodifluoromethane, dichlorotetrafluorethane, monochlorodigluoromethane, trichlorotrifluorethane, propane, butane, isobutane, dimethyl ether, carbon dioxide, nitrous oxide;

Solvents, such as ethyl alcohol, methylene chloride, isopropanol, castor oil, ethylene glycol monoethyl ether, diethylene glycol monobutyl ether, diethylene glycol monoethyl ether, dimethyl sulphoxide, tetrahydrofuran;

Humectants, such as glycerin, sorbitol, sodium 2-pyrrolidone-5-carboxylate, soluble collagen, dibutyl phthalate, gelatin; and

Powders, such as chalk, talc, fullers, earth, kaolin, starch, gums, colloidal silicon dioxide, sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate.

The amount of vehicle in the composition, including water if present, should preferably be sufficient to carry at least a portion of the active ingredient to the skin in an amount which is sufficient effectively to provide skin benefit. The amount of the vehicle can comprise the major portion of the composition, particularly where little or no other ingredients are present in the composition.

The composition will accordingly comprise from 15 to 99.989% and preferably from 50 to 99.5% by weight of the vehicle or vehicles.

The composition according the invention can contain ingredients other than those already mentioned, depending on the form of the intended product. It is, for example, possible to include antiseptics, preservatives, antioxidants, emulsifiers, colouring agents and detergents, some of which are described in detail above.

The composition according to the invention can also be employed as a vehicle for a wide variety of cosmetically or pharmaceutically active ingredients, particularly ingredients which have some beneficial effect when applied to the skin.

The composition thus provides a means whereby such active ingredients can be diluted, dispersed, conveyed to and distributed on the skin surface at an appropriate concentration.

The invention also provides a kit for skin care or treatment, comprising: a vessel containing the inventive composition, optionally further comprising instruction of how to use the inventive composition.

The inventive composition may also be embedded in a mask for the face or the body. The mask may comprise a backing sheet containing the inventive composition serving to exert a specific action on the skin. The backing sheet may be in a dry or web state, preferably stretchable at least in the wet state, in order to enable the mask to be adapted to fit the shape of the face or of the portion of the body to be treated. The backing sheet may be made of paper, fabric, cloth, or a polymeric material.

The invention also provides a process for the preparation of a cosmetic composition for topical application to skin which comprises mixing an active ingredient, as herein defined, with a suitable vehicle to provide a concentration of from 0.001% to 0.5%.

The compositions of the invention can be formulated as liquids, for example as a lotion or milk for use in conjunction with an applicator such as a roll-ball applicator, or a spray device such as an aerosol can containing propellant, or a container fitted with a pump to dispense the liquid product. Alternatively, the compositions of the invention can be solid or semi-solid, for example sticks, creams or gels, for use in conjunction with a suitable applicator or simply a tube, bottle or lidded jar, or as a liquid-impregnated fabric, such as a tissue wipe.

Preferably the composition is an aqueous emulsion and this can be a water-in-oil emulsion, or an oil-in-water emulsion. A particularly important composition or the invention is an aqueous fat emulsion in which the aqueous phase of the emulsion acts as a carrier.

Pharmaceutical compositions for topical application are particularly important, for skin condition is dependent on the presence of essential fatty acids, Such a composition can be liquid or plastic: liquid compositions include oils comprising the inventive composition with or without additional carrier oil; lotions, such as a solution in a physiologically acceptable solvent of an ester of the invention in free or derivative form, for instance an aqueous solution or an aqueous emulsion of the ester; and creams and ointments, such as a plastic dispersion of the ester in free or derivative form in a suitable carrier, for instance an ointment base. Such compositions are useful in the prevention and cure of skin damage caused by contact with detergents, and in treating environmental trauma due to weathering, sunburn, burns of other types and in reducing bacterial activity on the skin.

The invention accordingly also provides a closed container containing a cosmetic composition as herein defined.

Compositions of the invention are intended especially for topical application to human skin, in particular when the skin surface has become excessively dry, fissured, eroded or otherwise damaged.

The invention accordingly also includes a process of topical administration of the composition of the invention to human subjects suffering from or liable to suffer from excessively dry, fissured, eroded or otherwise damaged skin and other skin disorders. The dosage rate will depend on the condition to be treated as well as the route of administration. Local skin symptoms may require one or more applications of the composition.

The invention also provides for the use of an active ingredient, as herein defined, in the topical treatment of skin disorders.

The effectiveness of the inventive compositions on the promotion or restoration of the skin barrier function may be evaluated by using experimental animal models or tested on human subjects.

The model experimental animals usable in the present invention are those commonly used as experimental animals for various tests other than humans. Any animal may be used so long as in line with the object of the present invention, but usually rats, guinea pigs, rabbits, etc. are used.

The animals may be subjected to means for reducing the skin barrier function. The means may be any method capable of reducing the skin barrier function efficiently. The format of the means does not matter. To cause this reduction, it is convenient to remove the corneal layer of the epidermis by tape stripping or remove the corneal layer by treatment with a surfactant (for example, sodium dodecyl sulfate (SDS)) or an organic solvent such as acetone. As the timing of treatment, in the case of rats, e.g., after about 12 hours from the end of the application of the stress is preferable in ensuring a sufficient state of stress to the experimental animals.

The degree of recovery of the skin barrier function at the location of reduction of the skin barrier due to the above is detected over time or after a predetermined time interval. The location where the reduction is caused may be any location of the experimental animals so long as it enables detection of the degree of recovery of the skin barrier function, but usually it is preferable to select the auricle, that is, the projecting outer portion of the ear.

The method of detection may be to measure the transepidermal water loss or the transdermal insensible perspiration (TEWL)—generally considered an indicator of the skin barrier function, but it might be possible to use other factors capable of serving as indicators as well. The TEWL may be measured by a commercially available equipment itself in common use. As typical equipment, an Evaporimeter, a Tewameter, Micromoisture measurer, etc. may be mentioned.

For example, the effects of the inventive compositions on the promotion or restoration of skin barrier function can be tested on human subjects using an instrument for transepidermal water loss measurement, e.g., Tewameter TM-210 (Courage+Khazaka electronic, Cologne, Germany). For example, human subjects are allowed to rest and relaxed for 15-30 before the measurements started in an air-conditioned room, under constant temperature (21 degree celcius) and humidity 30-50%, with the skin at the measuring site left uncovered. The instrument is allowed to warm-up for 15 minutes after being turned on. The calibration is checked and the baseline value is set to zero once for all subsequent measurements. The measuring probe is placed perpendicular to the skin in a horizontal plane until stabilization of values is reached, about 60 seconds after the placement of the probe. Continuous holding by hand or any heat transfer to the probe is avoided. The contact pressure of the probe onto the skin is kept low and constant.

To test the effects of the inventive compositions on skin barrier function, experimental animals or human volunteers can be used. For example, on a human volunteer volunteer, several sites on the volar forearm are identified. The composition to be tested and placebo are applied randomly on those sites. At the determined time-points (varied according to the environment and subject conditions) the measurements of transepidermal water loss are taken. The data are tabulated and analyzed statistically.

Optionally, the effects of the inventive compositions on the promotion or restoration of skin barrier function can be tested on human subjects using an instrument for skin surface hydration measurement, e.g., Comeometer CM825 (Courage+Khazaka electronic, Cologne, Germany). For example, human subjects are allowed to rest and relaxed for 15-30 before the measurements started in an air-conditioned room, under constant temperature (21 degree celcius) and humidity 30-50%, with the skin at the measuring site left uncovered. The instrument is allowed to warm-up for 5 minutes after being turned on. The probe is wiped clean with a tissue paper soaked in 70% ethanol and zeroed against a dry clean surface (table or bench top) to establish the integrity of the instrument (reading should be less than 5). To obtain the reading 3 measurements are made within a skin skite and subsequently averaged. The probe is held by the handle only, gently placed perpendicular to the surface of the skin and depressed until the outer chamber of the probe comes in contact with the skin. The same pressure is exerted for all measurements, avoiding excess pressure. The instrument gives a beep sound when the measurement is complete. Before each subsequent measurement, the surface of the probe is wiped by the tissue paper soaked in 70% ethanol and the probe is re-zeroed against a dry surface. For example, on each volunteer, several sites on the volar forearm are identified. The formulation to be tested and placebo are applied randomly on those sites. At the determined time-points (varied according to the environment and subject conditions) the measurements of skin surface hydration are taken. The data are tabulated and analyzed statistically.

This present invention also provides a method for treating undesirable or pathological skin conditions of a mammal. The method comprises: topically applying to the skin of the mammal a composition comprising about 0.01-8% w/w divalent calcium ions and/or divalent magnesium ions based on the total weight of the composition in a physiologically acceptable medium for such period of time as required to elicit the desired cosmetic/therapeutic response. Examples of the undesirable skin condition include, but are not limited to, adverse signs of aging of the skin, a dull complexion, skin or hair pigmentation disorders, skin dryness, hyperseborrhoea, hyperseborrhoea-related imperfections, sensitive skin, dandruff, natural hair loss, and baldness. Examples of pathological skin condition include, but are not limited to, the dermatologic diseases linked to a keratinization disorder (differentiation-proliferation), inflammation and/or immunoallergy, such as acne vulgaris, blackheads or polymorphes, acne seniles, sunlight and medicinal or professional acne, rosacea, extensive and/or severe forms of psoriasis, and other keratinization disorders such as ichtyosis and ichtyosis-like states, Darier illness, palmoplantary keratodermies, leucoplasies and leucoplasie-like states, lichen plan, all benign or malignant dermatologic proliferations, extensive or severe, rheumatoid psoriases, pruritus; erythmas, atopic dermatitis, contact dermatitis, contact eczema, lichen planus, prurigo, urticaria, pruriginous toxidermia, and hyperkeratosis.

Each citation indicated above, whether of the open literature, patent, patent application, or otherwise, is hereby expressly incorporated by reference.

In order to further illustrate the present invention and the advantages thereof, the following specific examples are given, it being understood that same are intended only as illustrative and in nowise limitative.

In said examples to follow, all parts and percentages are given by weight.

### EXAMPLES

### Example 1

### Toner Solution

This example describes an embodiment of the compositions according to the invention, which is an aqueous solution and may be used as skin toner. The ingredients of the solution are listed as follows.

| | |
|---|---|
| Sodium chloride | 2.13% |
| Potassium chloride | 0.48% |
| Potassium bromide | 0.54% |
| Magnesium chloride | 2.02% |
| Calcium chloride | 2.06% |
| Glycerin | 1.02% |
| Water | 91.54% |
| Preservatives | 0.21% |

This solution contains combination of calcium ions with other salts in a composition to restore homeostatis of major ionic components in skin. The ratio of the monovalent ions: potassium and sodium from NaCl, KCl, KBr are balanced with divalent ions from MgCl₂ and CaCl₂. The mixture was prepared with attention not to cause “salting-out” phenomenon, in which less water-soluble salts were forced to crystallize after the addition of more water-soluble ones. The glycerin was used to maintain the viscosity of the solution.

Briefly, the solution in this example was prepared according to the following procedure. Sodium chloride, potassium chloride and potassium bromide were dissolved thoroughly into purified water by stirring. Thereafter, magnesium chloride and calcium chloride were added in a consecutive order while stirring until each one was totally dissolved. Glycerin and the preservative were then added. Before filling, the solution was passed through a 0.2-μm filter to rid of undissolved particles or other impurities.

### Example 2

### Skin Care Cream

This example describes an embodiment of the compositions according to the invention, which is a water-oil emulsion and may be used as skin care cream. The ingredients of the emulsion (designated as Cream Formulation I) are listed as follows.

| | |
|---|---|
| Pentaerythrityl tetracaprylate/tetracaprate | 5.2% |
| Emulsifying Wax | 3.9% |
| Behentrimonium methosulfate (and) cetearyl alcohol | 3.15% |
| Mineral Oil | 0.88% |
| Soybean Oil | 1.06% |
| Lanolin alcohol | 0.51% |
| Water | 81.72% |
| Calcium chloride | 0.22% |
| Glycerin | 1.9% |
| Wheat amino acids | 0.53% |
| Preservatives | 0.93% |
| Fragrance | q.s. |

In a variant of this embodiment, a water-oil emulsion was prepared with ingredients listed above except that the concentration of calcium chloride is reduced, and other divalent cations such as magnesium, as well as monovalent cations such as sodium and potassium, were added. The ingredients of this emulsion (designated as Cream Formulation II) are listed as follows.

| | |
|---|---|
| Pentaerythrityl tetracaprylate/tetracaprate | 5.2% |
| Emulsifying Wax | 5.89% |
| Behentrimonium methosulfate (and) cetearyl alcohol | 3.15% |
| Mineral Oil | 0.88% |
| Soybean Oil | 1.06% |
| Lanolin alcohol | 0.51% |
| Water | 79.58% |
| Calcium chloride | 0.104% |
| Magnesium chloride | 0.102% |
| Sodium chloride | 0.108% |
| Potassium chloride | 0.027% |
| Potassium bromide | 0.029% |
| Glycerin | 1.9% |
| Wheat amino acid | 0.53% |
| Preservatives | 0.93% |
| Fragrance | q.s. |

According to this embodiment of the invention, the water-oil emulsion provides calcium ions (which are optionally balanced with other divalent cations such as magnesium, as well as monovalent cations such as sodium and potassium), natural amino acids and essential skin lipid components. It is believed that by combining calcium ions with natural amino acids and/or essential skin lipid components, unwanted or adverse effects triggered by exogeneous calcium may be avoided. This is because applying calcium alone to the skin may force the skin to use low quality substitutes from the skin for building the skin barrier, especially when the skin is already damaged or in a pathological condition. Thus, the cream can supplement the skin with necessary “building blocks” for construction and restoration of the skin barrier.

In the cream, lanolin alcohol contains cholesterol, and the soybean oil provides fatty acids and short chain lipids essential to the skin. Wheat amino acids supply the materials necessary for naturally moisturizing the skin. The mixture of calcium chloride in glycerin-water is preferably added slowly to the lipid mixture at the designated temperature to avoid sudden precipitation of low molecular weight lipids by forming complex with calcium ions. The surfactants used have certain delivery capability to deliver the essential components to the target site in the skin.

Briefly, the cream in this example was prepared according to the following procedure. Pentaerythrityl tetracaprylate/tetracaprate, emulsifying wax, behentrimonium methosulfate and cetearyl alcohol, mineral oil, soybean oil and lanolin alcohol were added to a manufacturing vessel, and the vessel was stirred and heated to 75° C. Calcium chloride was dissolved into water and glycerin was then added. The aqueous solution was heated to 75° C. and added into the manufacturing vessel with quick stirring, resulting in a mono-phase emulsion. The vessel was cooled down to 40° C. When the desired temperature was reached, wheat amino acids, preservatives and fragrance was added and mixed thoroughly in the vessel.

### Example 3

### Skin Care Gel

This example describes an embodiment of the compositions according to the invention, which is a silicone-based gel and may be used as skin care gel. The ingredients of the gel are listed as follows.

| | |
|---|---|
| Cyclopentasiloxane (and) dimethicone crosspolymer | 70.44% |
| (and) cyclohexasiloxane | |
| Cyclomethicone | 19.52% |
| Propylene glycol | 3.64% |
| Soluble collagen | 0.49% |
| Hydrolized silk protein | 0.72% |
| Tocopheryl acetate | 0.45% |
| Glycerin | 2.98% |
| Marine collagen amino acids | 0.53% |
| Calcium chloride | 0.21% |
| Preservatives | 1.02% |

This formulation combines calcium ions with amino acids for enhancing collagen production and anti-aging properties. It is believed that adding exogenous calcium alone may not be sufficient for promoting collagen production in skin. The soluble collagen in the gel is intended to give direct supply of such an essential skin component whereas the collagen amino acids supply the building blocks for synthesis of collagen and elastin. In this formulation it is preferred that no water was added, although moderate amount of water should not be harmful.

Briefly, the gel in this example was prepared according to the following procedure. The mixture of cyclopentasiloxane, dimethicone crosspolymer, cyclohexasiloxane (e.g., Silicone Elastomer Blend) was added to cyclomethicone and stirred in a manufacturing vessel. Propylene glycol, soluble collagen, hydrolized silk protein, tocopheryl acetate, glycerin, marine collagen amino acids, calcium chloride and preservatives were mixed in separate vessel, and then added into the manufacturing vessel with continuous moderate stirring. Care was taken with stirring to avoid too much aeration.

### Example 4

### Skin Cleansing Lotion

This example describes an embodiment of the compositions according to the invention, which may be used as skin cleanser. The ingredients of the skin cleansing lotion are listed as follows.

| | |
|---|---|
| Water | 69.95% |
| Decyl Oleate | 2.28% |
| Sodium Lauryl Sulfate | 4.96% |
| Stearic Acid | 3.18% |
| Lauramide DEA | 1.25% |
| Glyceryl Stearate S.E. | 2.47% |
| Cetyl Alcohol | 1.98% |
| Propylene glycol | 2.37% |
| Triethanolamine | 0.24% |
| Phospholipids | 0.82% |
| 1,3-Butylene glycol | 0.69% |
| Cholesterol | 0.36% |
| Tocopheryl Acetate | 0.21% |
| Thiamine Mononitrate | 0.09% |
| Niacin | 0.11% |
| Calcium D-Panthothenate | 0.12% |
| Pyridoxine | 0.04% |
| Ascorbic Acid | 0.12% |
| Retinyl Palmitate | 0.05% |
| Cholecalciferol | 0.06% |
| Phytonadione | 0.09% |
| Polysorbate-80 | 8.11% |
| Diazolidinyl Urea | 0.29% |
| Methyl paraben | 0.11% |
| Propyl paraben | 0.05% |

The inventors believe that the use of skin cleansers with excessive solubilization strength can extract out water-soluble components out of the skin, including ions of calcium, magnesium, sodium, potassium, and many others. To prevent such extraction of the vital minerals, the inventive cleanser is provided, which, when applied to the skin and washed off, leaves a thin fatty layer on the surface of the skin after cleansing. Such a layer is formed by incorporating fatty film forming ingredients in the cleanser such as Decyl Oleate, Lauramide DEA, and Glyceryl Stearate S.E.

### Example 5

### In Vitro Study to Assess Change of Calcium Ion Distribution in Epidermis

This example describes an in vitro study undertaken to assess the change of the calcium ion distribution in epidermis before and after the application of the toner formulation described in Example 1 and Cream Formulation II in Example 2.

1) Materials and Methods

Standards: Calcium chloride in 20% (w/v) gelatin dissolved in hot, distilled water with 5% glycerin. Standards were quench frozen (in Freon) at approximately −190° C. and cryosectioned (at −20° C.) to a thickness of 12-16 μm, then they were freeze-dried

Samples: Fresh human abdominal skin membrane was obtained from surgery. After removal of fat, the skin membrane was washed using 10% aqueous sodium lauryl sulfate solution for 3 hours at 25° C. to deprive the minerals. After dried in the air, the skin was cut into several pieces. Some pieces were left untreated. The epidermal surface of the remaining pieces was treated for 30 minutes with the subsequent application of the toner formulation described in Example 1 and Cream Formulation II in Example 2. Then, the surface was dried with tissue paper to remove excessive materials. All samples (treated and untreated) were quench frozen (in Freon) at approximately −190° C. and cryosectioned (at −20° C.) to a thickness of 15-20 μm, then they were freeze-dried.

Analysis: Analysis was performed using Proton Induced X-Ray Emission (PIXE) Method. Samples were mounted between two supporting foils. Proton of 2.5 MeV from an electrostatic accelerator (Pelletron 3UDH) were collimated and focused by quadrupole magnets to form a rectangular beam spot at the sample of 5-10×100 μm². The proton current density was 1.2 pA/μm². The total charge accumulated in each analysis was 0.1 μC and the X-rays were detected in a Kevex Si(Li) detector (80 mm², internally collimated to 60 mm², FWHM=160 eV at 5.9 keV).

2) Results

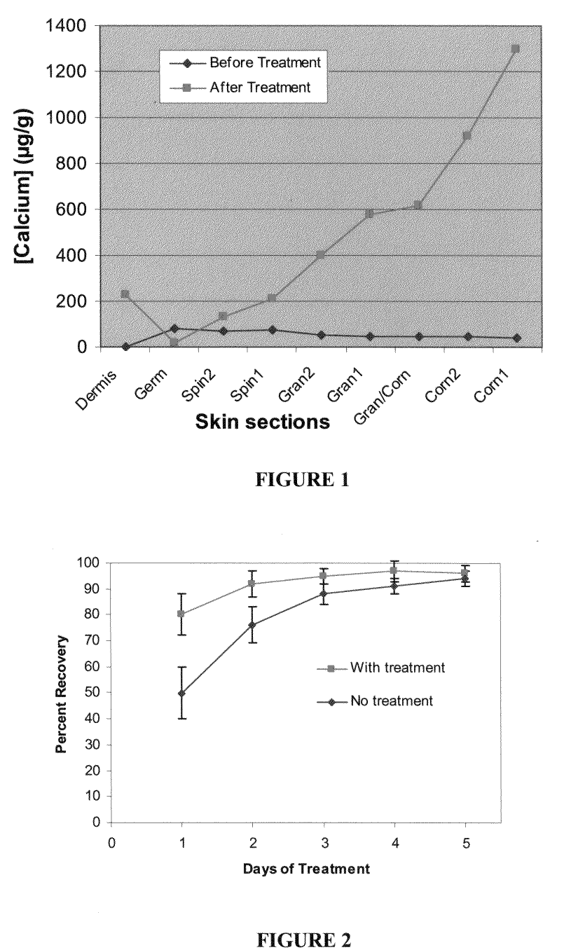

The calcium concentration in each skin sections was assessed. As shown in FIG. 1, the untreated skin sections (“Before treatment”) showed low calcium concentration. The treated sections (“After treatment”) showed a concentration gradient of calcium ions which was higher at the upper layers (close to the skin surface) and lower in the deeper epidermis. This pattern of calcium concentration gradient is very similar to the pattern reported in the literature for normal skin. See Malmqvist et al. (1987) “The use of PIXE in experimental studies of the physiology of human skin epidermis”, Biol. Trace Elem. Res. 12:297-308; and Pallon et al. (1996) “Pixe analysis of pathological skin with special reference to psoriasis and atopic dry skin,” Cell Mol Biol (Noisy-le-grand) 42 (1):111-8.

3) Conclusion

This in vitro study showed that the inventive toner and cream can deliver calcium ions into skin and achieve the restoration the calcium concentration gradient of mineral deprived skin membranes to the pattern found in normal skin.

### Example 6

### In Vivo Study to Assess Effects of Inventive Toner and Cream on Restoration of Skin Barrier Function

This example describes an in vitro study undertaken to assess the effects of the toner formulation described in Example 1 and Cream Formulation II in Example 2 on the restoration of skin barrier function by using Transepidermal Water Loss (TEWL) measurements.

1) Materials and Methods

Five human subjects with normal skin, with age ranging from 25 to 45, participated in the study. The TEWL measurements were performed using Tewameter TM-210 (Courage+Khazaka, Germany) on two volar forearm sites of the same arm each subject. After a period of acclimatization, TEWL measurements were taken to assess the baseline values. The sites were intentionally damaged by stripping 15 times using Scotch Tape, but not fully removing the epidermis. One site was left untreated but covered with gauze. The other site was treated with the subsequent application of the skin toner described in Example 1 and Cream Formulation II in Example 2 twice a day with 8 hour interval. Once a day prior to the treatment, the TEWL on the sites were measured. This was repeated up to 5 days after the tape stripping.

2) Results:

FIG. 2 showed the progress of skin barrier restoration, calculated as the TEWL baseline values/TEWL after the stripping and treatments. Without treatments, living human skin showed capacity to recover. The treatment of the inventive toner and cream accelerated the recovery process in the first 3 days after the skin damage by stripping.

3) Conclusion

Application of the inventive toner and cream can accelerate barrier restoration compared to the site without application.

### Example 7

### In Vivo Study to Assess Effects of Inventive Toner and Cream on Human Subjects with Known Condition of Dry Skin

This example describes an in vivo study undertaken to assess the effect of the toner formulation described in Example 1 and Cream Formulation II in Example 2 on subjects with known condition of dry skin.

1) Materials and Methods

A total of 30 subjects, male and female aged 17 to 60 years, with minimal to mild dry skin symptoms in low leg area, participated in the study. Each subject was given a set of toner formulation described in Example 1 and Cream Formulation II in Example 2 to be applied to the symptomatic area twice daily for two weeks. The daily self-evaluation was documented on the score of scaling and/or erythema on the Clinical Research Form (CRF). On week 2 the CRF was collected from the subjects for data analysis.

Safety evaluation: Safety was assessed from vital signs, signs and symptoms of applied skin, and reported adverse experiences.

Efficacy evaluation: To determine the efficacy, the time to reach no clinical sign of scaling was used as a parameter.

2) Results:

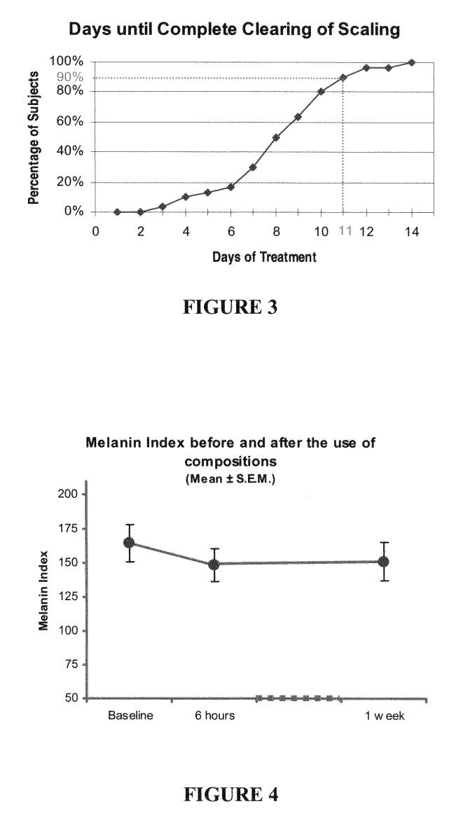

Based on basic scoring for scaling, all subjects had conditions that fell below the scale of 2, which is mild; fine, flaky scale predominated. The average score was 1.78 for 30 subjects. Age median was 32.5 years old. On safety evaluation, all subjects reported no adverse effects during the period of the study. The analysis of the Clinical Research Form has shown the time to reach no clinical sign of scaling for each subject as tabulated below and plotted in FIG. 3. It was observed that 90% subjects reported no clinical sign of scaling after 11 days of using the inventive toner and cream as instructed.

| | Number | Cumulative | |
|---|---|---|---|
| Days of | of | No. | Percentage |
| Treatment | subjects | Subjects | of Subjects |
| 1 | 0 | 0 | 0% |
| 2 | 0 | 0 | 0% |
| 3 | 1 | 1 | 3% |
| 4 | 2 | 3 | 10% |
| 5 | 1 | 4 | 13% |
| 6 | 1 | 5 | 17% |
| 7 | 4 | 9 | 30% |
| 8 | 6 | 15 | 50% |
| 9 | 4 | 19 | 63% |
| 10 | 5 | 24 | 80% |
| 11 | 3 | 27 | 90% |
| 12 | 2 | 29 | 97% |
| 13 | 0 | 29 | 97% |
| 14 | 1 | 30 | 100% |
| Total | 30 | | |

3) Conclusion

The study demonstrates the safety and efficacy of the inventive toner and cream to alleviate the symptoms of dry skin in minimal to mild cases. No adverse reaction was reported. 90% of subjects reported clearing of scaling after 11 days using the products.

### Example 8

### In Vivo Study to Assess Effects of Inventive Toner and Cream on Melanin Production and Hyperpigmentation Prevention

This example describes an in vivo study undertaken to assess the effects of the toner formulation described in Example 1 and Cream Formulation II in Example 2 on the melanin index of facial skin.

1) Materials and Methods

Eight human subjects with mild dry skin participated in the study. The melanin index was measured using a Mexameter (Courage+Khazaka, Germany). After the baseline melanin index measurement taken, the skin toner described in Example 1 and Cream Formulation II in Example 2 was applied to the subjects' face twice a day for one week. The melanin index measurements were performed 6 hours and 1 week after initial application.

2) Results

FIG. 4 shows the progress of melanin index. Application of the composition on subjects with mild dry skin showed a decrease of melanin index compared to a normalized condition, starting 6 hours after initial application. Upon using the compositions twice daily for 1 week, the melanin index remained at normalized condition.

3) Conclusion

Application of the inventive toner and cream can normalize melanin production to prevent hyperpigmentation. Application can also improve the skin barrier as melanin index reduction can be an indirect effect of skin barrier improvement.

### Example 9

### In Vivo Study to Assess Effects of Inventive Cream on Facial Skin Moisture Content and Maintenance

This example describes an in vivo study undertaken to assess the effects of the toner formulation described in Example 1 and Cream Formulation II in Example 2 on facial skin moisture by using Transepidermal Water Loss (TEWL) measurements.

1) Materials and Methods

Eight human subjects with mild dry skin participated in the study. The TEWL measurements were performed using Tewameter TM-210 (Courage+Khazaka, Germany) on the face of each subject. After a period of acclimatization, TEWL measurements were taken to assess the baseline values. The face was treated with application of the skin toner described in Example 1 and Cream Formulation II in Example 2 twice daily for 6 hours, 1 week, or up to 2 weeks, respectively. The TEWL was measured 6 hours, 1 week, and 2 weeks after initial application

2) Results

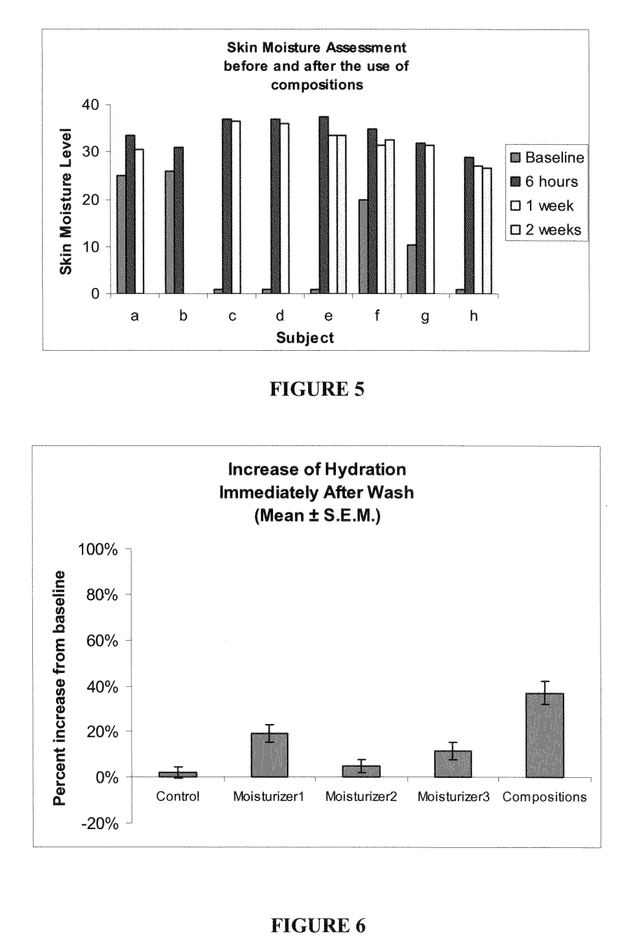

FIG. 5 shows the progress of skin barrier restoration, calculated as the TEWL baseline values/TEWL after treatments. The treatment of the inventive toner and cream on the mild dry skin showed improved moisture content 6 hours after initial treatment (before application) and the elevated moisture content (compared to baseline) was sustained for all subjects during the experimental period.

3) Conclusion

Application of the inventive toner and cream can increase and maintain skin moisture content.

### Example 10

### In Vivo Study to Assess Effects of Inventive Cream on Skin Moisture Content After Washing

This example describes an in vivo study undertaken to assess the effects of the toner formulation described in Example 1 and Cream Formulation II in Example 2 on the skin moisture content after washing using Corneometer measurements.

1) Materials and Methods

Ten human subjects of both genders, with age range 20 to 50 years old, participated in the study. Measurements for skin moisture content were performed using the Corneometer for skin moisture content (Courage+Khazaka, Germany) on the volar forearm sites of the subject. The application skin site was free from visual blemishes. Baseline values were obtained prior to application of the compositions of the toner formulation described in Example 1 and Cream Formulation II in Example 2. The baseline readings of skin moisture content were within the normal values. The compositions were applied on the skin for 1 hour and then washed away with water and dried with paper towel. The measurements were performed 5 minutes afterwards. Three other moisturizers were also used for comparison.

2) Results

FIG. 6 shows the increased moisture content of the skin. After washing all applications from the skin surface, the moisture content increase on the site treated with the toner formulation described in Example 1 and Cream Formulation II in Example 2 was still 40% greater than the baseline, whereas the other moisturizers provided an increase in skin moisture of up to 20%.

3) Conclusion

Application of the inventive toner and cream to increase skin moisture and hydration can be maintained after washing. Higher moisture content can indicate improved skin barrier.

### Example 11

### In Vivo Study to Assess Effects of Inventive Cream on Skin Elasticity After Washing

This example describes an in vivo study undertaken to assess the effects of the toner formulation described in Example 1 and Cream Formulation II in Example 2 on skin elasticity after washing using Cutometer measurements.

1) Materials and Methods

Ten human subjects of both genders, with age range 20 to 50 years old, participated in the study. Measurements for skin elasticity were performed using the Cutometer for skin elasticity (Courage+Khazaka, Germany) on the volar forearm sites of each subject. The application skin site was free from visual blemishes. Baseline values were obtained prior to application of the compositions of the toner formulation described in Example 1 and Cream Formulation II in Example 2. The baseline readings of skin elasticity were within the normal values. The compositions were applied on the skin for 1 hour and then washed away with water and dried with paper towel. The measurements were performed 5 minutes afterwards. Three other moisturizers were also used for comparison.

2) Results

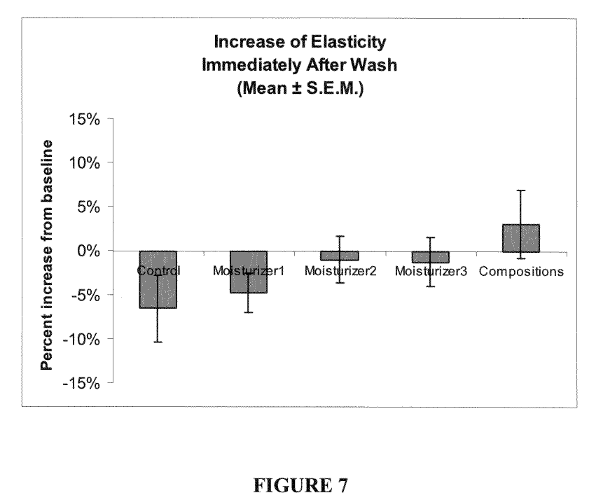

FIG. 7 shows the elasticity of the skin after washing all applications from the skin surface. The elasticity increased on the site treated with the toner formulation described in Example 1 and Cream Formulation II in Example 2, whereas sites treated with other moisturizers showed decreased elasticity.

3) Conclusion

Application of the inventive toner and cream can increase skin elasticity and the increase in skin elasticity can be maintained after washing. Elevated skin elasticity can indicate improved water content and skin barrier.

### Example 12

### In Vivo Study to Assess Effects of Inventive Cream on Skin Barrier Recovery Post CO2 Laser Resurfacing

This example describes an in vivo study undertaken to assess the effects of the toner formulation described in Example 1 and Cream Formulation II in Example 2 on skin barrier recovery after CO2 laser resurfacing.

1) Materials and Methods

The CO2 laser resurfacing procedure was performed on subject. One day after procedure, the toner formulation described in Example 1 and Cream Formulation II in Example 2 was applied three times a day. Photos were taken 1 day after the procedure and 6 days after procedure.

2) Results

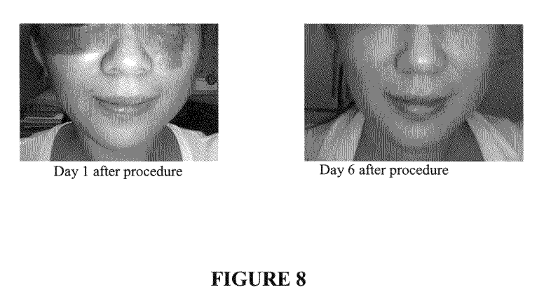

FIG. 8 shows the subject one day after the CO2 laser resurfacing procedure and 6 days after the procedure with application of the toner formulation described in Example 1 and Cream Formulation II in Example 2. The subject's skin recovered after 6 days.

3) Conclusion

Application of the inventive toner and cream can accelerate skin barrier restoration post CO2 laser resurfacing, from an expected recovery time of about 2-4 weeks to about 6 days.

### Example 13

### In Vivo Study to Assess Effects of Inventive Cream on Skin Barrier Recovery Post Ruby Laser Treatment

This example describes an in vivo study undertaken to assess the effects of the toner formulation described in Example 1 and Cream Formulation II in Example 2 on skin barrier recovery after ruby laser treatment.

1) Materials and Methods

Ruby laser treatment was performed on subject for treatment of hyperpigmentation. One day after procedure, the toner formulation described in Example 1 and Cream Formulation II in Example 2 was applied twice a day. Photos were taken 1 day, 6 days, and 12 days after procedure.

2) Results

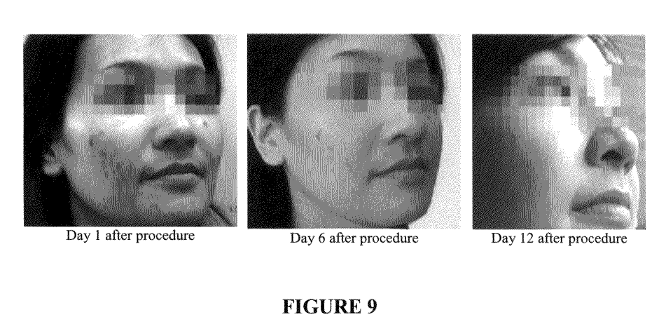

FIG. 9 shows the subject one day after the ruby laser treatment, and 6 and 12 days after the procedure with application of the toner formulation described in Example 1 and Cream Formulation II in Example 2. The subject's skin recovered after 12 days without common side effects such as post-inflammatory hyperpigmentation.

3) Conclusion

Application of the inventive toner and cream can accelerate skin barrier restoration post ruby laser treatment, from an expected recovery time of about 10-20 days to about 6-12 days.

What is claimed is:

1. An aqueous composition, comprising:
divalent calcium cations, divalent magnesium cations, chloride anions, and potassium bromide wherein the composition comprises about 0.01-8% w/w divalent calcium cations, and about 0.01-8% w/w divalent magnesium cations based on the total weight of the composition in a physiologically acceptable medium, and the weight ratio of divalent calcium chloride to potassium bromide ranges from about 6:1 to 1:5.

2. The composition according to claim 1, wherein the composition is an aqueous solution comprising at least 80% w/w water.

3. The composition according to claim 1, wherein the weight ratio of calcium chloride to magnesium chloride ranges from about 2:1 to 1:3.

4. The composition according to claim 1, wherein the composition further comprises sodium monovalent cations, and the ratio of divalent calcium ions to monovalent cations ranges from 15:1 to 1:20.

5. The composition according to claim 1, wherein the composition further comprises sodium monovalent cations, and the ratio of divalent calcium ions to monovalent cations ranges from 4:1 to 1:3.

6. The composition according to claim 1, further comprising sodium ions.

7. The composition according to claim 6, wherein the sodium ions are provided by sodium chloride.

9. The composition according to claim 7, wherein the composition comprises calcium chloride and sodium chloride at a weight ratio ranging from 5:1 to 1:10.

10. The composition according to claim 7, wherein the composition comprises calcium chloride and sodium chloride at a weight ratio ranging from 2:1 to 1:3.

11. The composition according to claim 7, wherein the composition comprises calcium chloride and sodium chloride at a weight ratio of about 1:1.

12. The composition according to claim 11, wherein the composition further comprises potassium chloride and the weight ratio of calcium chloride to potassium chloride ranges from 10:1 to 1:5.

13. The composition according to claim 11, wherein the composition further comprises potassium chloride and the weight ratio of calcium chloride to potassium chloride is about 4:1.

14. The composition according to claim 13, wherein the weight ratio of calcium chloride to magnesium chloride ranges from about 2:1 to about 2:3.

15. The composition according to claim 13, wherein the weight ratio of calcium chloride to magnesium chloride is about 1:1.

17. The composition according to claim 14, wherein the weight ratio of calcium chloride to potassium bromide is about 4:1.

18. The composition according to claim 1, wherein the composition is in a form of emulsion.

19. The composition according to claim 18, wherein the amount of divalent calcium ion is about 0.1-0.3%.

20. The composition according to claim 18, further comprising: amino acids in an amount of 0.002-6% w/w based on the total weight of the composition.

21. The composition according to claim 1, wherein the composition is in the form of a lotion, a gel, a water-in-oil emulsion, an oil-in-water emulsion, a triple emulsion, nanocapsules, liposomes, nanoemulsions, or oleosomes.

22. A method for treating, inhibiting or preventing inflammation of the skin of a subject, comprising:
topically applying to the skin of the subject a composition comprising divalent calcium ions, divalent magnesium ions, chloride, and potassium bromide wherein the composition comprises about 0.01-8% w/w divalent calcium ions, and about 0.01-8% w/w divalent magnesium ions based on the total weight of the composition in a physiologically acceptable medium, and the weight ratio of divalent calcium chloride to potassium bromide ranges from about 6:1 to 1:5.

23. The method according to claim 22, wherein the composition is in the form of aqueous solution comprising at least 80% of water and further comprises sodium monovalent cations, and the ratio of divalent calcium ions to sodium monovalent cations ranges from 5:1 to 1:3 by weight.

24. The method according to claim 23, further comprising:
topically applying an emulsion comprising at least 70% of water and divalent calcium cations and monovalent cations at a ratio ranging from 8:1 to 1:2 by weight.

25. The method according to claim 24, wherein the calcium ions are provided by calcium chloride; and monovalent cations are sodium or potassium ions provided by sodium chloride, potassium chloride or potassium bromide.

26. The method according to claim 22, wherein the inflammation of the skin of the subject is caused by a medical, dermotological or cosmetic procedure performed on the subject.

27. The method according to claim 26, wherein the procedure is selected from the group consisting of chemical peeling, laser skin resurfacing, hair removal using chemicals or light energy, vein removal, microdermabrasion, plasma energy treatment, radiofrequency treatment, skin treatment using light energy, and photofacial.

28. The method according to claim 27, wherein the light energy is at a wavelength of about 200 nm to 10000 nm.

29. The method according to claim 22, wherein the inflammation of the skin causes hyperpigmentation of the skin.

30. The method according to claim 26, wherein the skin barrier function of the subject is restored at least 2-15 days faster than the time required for restoration of the skin barrier function of another subject who has gone through the same cosmetic procedure but without being treated with the composition.

31. The method according to claim 26, wherein the skin barrier function of the subject is restored at least 5-12 days faster than the time required for restoration of the skin barrier function of another subject who has gone through the same cosmetic procedure but without being treated with the composition.

32. The method according to claim 26, wherein the skin barrier function of the subject is restored 5-14 days faster than the time required for restoration of the skin barrier function of another subject who has gone through the same cosmetic procedure but without being treated with the composition.

33. The method according to claim 26, wherein the inflammation of the skin of the subject is reduced at least 50% within a time period at least 2-15 days shorter than the time required for at least 50% reduction of inflammation of the skin of another subject who has gone through the same cosmetic procedure but without being treated with the composition.

35. The method according to claim 26, wherein the inflammation of the skin of the subject is reduced at least 50% within a time period at least 5-12 days shorter than the time required for at least 50% reduction of inflammation of the skin of another subject who has gone through the same cosmetic procedure but without being treated with the composition.

36. The method according to claim 26, wherein the inflammation of the skin of the subject is reduced at least 50% within a time period that is 5-14 days shorter than the time required for at least 50% reduction of inflammation of the skin of another subject who has gone through the same cosmetic procedure but without being treated with the composition.

37. The method according to claim 26, wherein the composition is administered topically to the subject prior to the procedure.

38. The method according to claim 26, wherein the composition is administered topically to the subject at least 1-10 days prior to the procedure.

39. The method according to claim 26, wherein the composition is administered topically to the subject at least 1-60 minutes after the procedure.

40. A kit, comprising: a vessel comprising a composition comprising
divalent calcium cations, divalent magnesium cations, chloride anions, and potassium bromide wherein the composition comprises about 0.01-8% w/w divalent calcium cations, and about 0.01-8% w/w divalent magnesium cations based on the total weight of the composition in a physiologically acceptable medium, and the weight ratio of divalent calcium chloride to potassium bromide ranges from about 6:1 to 1:5.

41. The kit according to claim 37, further comprising: a printed instruction for using the composition for cosmetic purposes.

42. A system for skin treatment using light energy, comprising:
a source of light energy for treating the skin of a subject; and
a composition comprising divalent calcium cations, divalent magnesium cations, chloride anions, and potassium bromide wherein the composition comprises about 0.01-8% w/w divalent calcium cations, and about 0.01-8% w/w divalent magnesium cations based on the total weight of the composition in a physiologically acceptable medium, and the weight ratio of divalent calcium chloride to potassium bromide ranges from about 6:1 to 1:5.
